(19) United States
(12) Patent Application Publication
(10) Pub. No.: US 2007/0207531 A1
(43) Pub. Date: Sep. 6, 2007

(54) METHODS AND BACTERIAL STRAINS FOR PRODUCING HYDROGEN FROM BIOMASS

(76) Inventors: Mongi Ferchichi, Richmond (CA); Amer Almadidy, Surrey (CA); Edward Crabbe, Surrey (CA); Gwanghoon Gil, Surrey (CA); William Hintz, Victoria (CA)

Correspondence Address: KLARQUIST SPARKMAN, LLP, 121 SW SALMON STREET, SUITE 1600, PORTLAND, OR, 97204, US

(21) Appl. No.: 11/570,038

(22) PCT Filed: May 26, 2005
(86) PCT No.: PCT/CA05/00806

§ 371 Date: Dec. 4, 2006

Related U.S. Application Data
(60) Provisional application No. 60/576,627, filed on Jun. 4, 2004.

(51) Int. Cl. C12P 3/00; C12N 15/74; C12N 1/21

(52) U.S. Cl. 435/168; 435/252.3; 435/471

(57) ABSTRACT

A method is provided for producing hydrogen by fermenting a culture medium containing a sugar and maintained under substantially anaerobic conditions with a bacterium of the genus Clostridium. The bacterium may be Clostridium bifermentans and hydrogen may be produced with an efficiency of at least about 34% relative to the maximum theoretical possible yield. There are also disclosed substantially pure cultures of bacteria of the genus Clostridium which can ferment sugars present in a culture medium at a concentration of between about 1% and 10% under substantially anaerobic conditions to produce hydrogen with an efficiency of at least about 34% of the maximum possible theoretical yield

### FIELD OF THE INVENTION

The present invention relates to bacteria for the production of hydrogen by fermentation of biomass, the use of such bacteria and methods for the production of hydrogen and treatment of biomass by such bacteria.

### BACKGROUND OF THE INVENTION

Among the alternatives to fossil fuels, hydrogen gas is relatively clean and has a high energy yield (Kovacs et al. (2000) Recent advances in biohydrogen research. Eur. J. Physiol. 439 [Suppl]:R81-R83.). Unfortunately, many methods for the synthesis of hydrogen may only be practical where electricity is very inexpensive (Rajeshwar et al., (1994) Electrochemistry and the environment. J. Appl. Electrochem. 24:1077-1091) or may themselves use fossil fuels to produce the hydrogen gas.

Anaerobic digestion of organic waste produces an abundance of methane which is a fuel source, but its combustion product (CO₂) is still a greenhouse gas. It is therefore desirable to produce hydrogen gas rather than methane by such fermentation procedures. Plant cell wall materials from the pulp and paper industry, being primarily composed of energy-rich carbohydrates such as hemicellulose, cellulose and lignin, are an attractive renewable resource for the production of hydrogen. Several other biomass sources have also been suggested as fuel sources, including wastewater containing readily fermentable sugars, the organic fraction of municipal solid waste, sugar cane juice, and corn pulp (Pipatti et al., (1995) Greenhouse impacts of anthropogenic CH₄ and N₂O emissions in Finland. Environ. Mgmt. 19:561-567).

Some bacterial species known to produce hydrogen include Clostridium, Escherichia, Citrobacter, and Bacillus (Lay et al., (1999) Feasibility of biological hydrogen production from organic fraction of municipal solid waste. Water Res. 33: 2579-2586; Nandi & Sengupta, (1998) Microbialproduction of hydrogen: an overview. Crit. Rev. Microbiol. 24:61-84). U.S. Pat. No. 4,480,035 to Roychowdhury, issued Oct. 30, 1984, describes the use of Citrobacter freundii, Enterobacter aerogenes, Escherichia coli, bacillus and pseudomonas to aerobically ferment glucose generated by hydrolysis of cellulosic waste; U.S. Pat. No. 6,409,841 to Lombard, issued Jun. 25, 2002, describes bacterial fermentation of free sugars and oligosaccharides derived from lignocellulose containing biomass. Hydrogen fermentation may be able to use the same hardware commonly used for methane fermentation (van Ginkel et al., (2001), Biohydrogen production as a function of pH and substrate concentration. Environ. Sci. Technol. 35:4726-4730.) but economically viable production of hydrogen from mixed cultures may require the suppression of the hydrogen consuming bacteria (Lay (2000), Modeling and optimization of anaerobic digested sludge converting starch to hydrogen. Biotechnol. Bioeng. 68:269-278).

### SUMMARY OF THE INVENTION

According to one embodiment, there is disclosed a method for producing hydrogen comprising fermenting a culture medium comprising at least one sugar and maintained under substantially anaerobic conditions with a bacterium selected from the genus clostridium to produce hydrogen with an efficiency of at least about 34% relative to the maximum theoretically possible yield.

According to a further embodiment, there is disclosed a method for the treatment of biomass comprising treating the biomass to liberate sugars and fermenting the sugars with a substantially pure culture of a bacteria of the genus clostridium maintained under substantially anaerobic conditions to produce hydrogen with an efficiency of at least about 34% relative to the theoretical maximum possible yield.

According to a further embodiment, there is disclosed the use of a species of bacterium of the genus Clostridium to ferment a culture medium comprising at least one sugar and maintained under substantially anaerobic conditions, to produce hydrogen at an efficiency of at least about 34% of the theoretically possible maximum yield.

According to a further embodiment, there is disclosed a substantially pure culture of one or more species of bacteria of the genus Clostridium which can ferment glucose present in a culture medium at a concentration of between about 1% and about 10% under substantially anaerobic conditions to produce

According to a further embodiment, there is disclosed a substantially pure culture of one or more species of bacteria of the genus Clostridium which can ferment lactose present in a culture medium at a concentration of between about 1% and 10% under substantially anaerobic conditions to produce hydrogen with an efficiency of at least about 34% of the maximum possible theoretical yield.

According to further embodiments, there are disclosed the methods, uses, or cultures wherein the efficiency is at least about 38%.

According to further embodiments the efficiency is at least about 42%.

According to further embodiments the fermentation occurs at a temperature between about 30° C. and about 50° C.

According to further embodiments the fermentation occurs at a temperature between about 50° C. and about 70° C.;

According to further embodiments the mixture has an initial pH of between about 6.5 and about 8.5.

According to further embodiments the sugar is present at a concentration of between about 0.5% and about 8.0%, or between about 0.5% and 8.0%.

According to further embodiments the mixture is held at a pH of between about 6.5 and about 8.0 during the fermentation or at a pH of between about 6.5 and about 8.5 during fermentation.

According to further embodiments the culture medium is supplemented with a suitable nitrogen source.

According to further embodiments the bacterium is selected from the group consisting of IDA deposits KAM 1, KAM 5, KAM 7, KAM 8, KAM 10, KAM 12 and KAM 13.

According to further embodiments the bacterium is descended from the bacterium of any of the group of bacteria comprising IDA deposits KAM 1, KAM 5, KAM 7, KAM 8, KAM 10, KAM 12 and KAM 13.

According to further embodiments the sugar is selected from the group consisting of maltose, sucrose, fructose, galactose, arabinose, lactose, glucose, xylose, mannose and mixtures thereof.

According to further embodiments the mixture comprising sugars is derived from biomass. According to further embodiments the biomass has a high carbohydrate content, the biomass comprises agricultural waste, the biomass comprises food processing waste, the biomass comprises industrial waste, the biomass comprises cellulose or lignocellulose, the biomass comprises pulp and paper mill waste or newspaper print fiber, or the biomass comprises, dairy waste, waste meat, corn cobs, nut shells, husks, sugar cane bagasse, agro-industrial waste fiber, spoilt milk, cheese, whey, apple processing waste, raspberry processing waste, brewing yeast residues, and mixtures thereof.

Further embodiments comprise treating the biomass to liberate free sugars.

Further embodiments comprise mechanically fragmenting the biomass.

Further embodiments comprise; (a). enzymatically hydrolysing a component of the biomass; or (b). acidically hydrolysing a component of the biomass; or (c). enzymatically and acidically hydrolysing a component of the biomass to release free sugars.

According to further embodiments, the maintaining of the substantially anaerobic conditions comprises sparging the culture medium with nitrogen.

According to further embodiments the sugar is glucose or lactose.

According to further embodiments the bacterium is Clostridium difficile, or the bacterium is Clostridium bifermentans.

### BRIEF DESCRIPTION OF THE FIGURES

FIG. 1: Schematic depiction of the experimental protocol for monitoring hydrogen production from waste materials.

FIG. 2: Graph showing anaerobic hydrogen production by Clostridium beijerinckii (ATCC 10132).

FIG. 3: Graph showing hydrogen gas production by Clostridium beijerinckii (ATCC 10132).

### DETAILED DESCRIPTION OF THE PREFERRED EMBODIMENTS

In this application the term “anaerobic” has its ordinary meaning and refers to conditions in which there is little or no free oxygen available. With respect to a natural environment it refers to one where there is little or no free oxygen and may include but is not limited to flower garden soils, bogs, river soils, potato field soils, wounds, guts of animals, refuse, contaminated food and water, waste materials, effluent, and otherwise artificially or naturally oxygen depleted environments.

In this application “biomass” means any source of carbohydrate and includes but is not limited to waste materials. It may be solid, semi-solid, liquid, effluent, a suspension or solution, or may have any other physical forms.

In this application “carbohydrate” has its normal meaning and refers to any of the of the large group of energy-producing organic compounds with the general formula CₙH₂ₙOₙ. It may have straight chain, branched chain or ring configurations and may be substituted or unsubstituted, complexed or uncomplexed and includes carbohydrates in free form or combined with proteins, lipids and other organic or inorganic compounds. It includes mono, di, tri, oligo and polysaccharides and polymers, typical examples include but are not limited to starch and cellulose.

The term “Clostridium” refers to any members of the group of bacteria generally referred to by this name and characterized in that its members may be gram-positive, spore-forming, rod shaped and anaerobic. Four examples of the genus Clostridium are: C. tetani, C. difficile, C. perfringens, and C. botulinuin and further examples are to be found as ATCC deposit numbers ATCC 27021, and ATCC 10132.

In this application, the term “efficiency” means, when used regarding hydrogen production, the actual hydrogen yield in moles of hydrogen per mole of the relevant carbon source and is expressed as a percentage of the theoretical maximum possible yield (for example, with glucose, a 100% yield indicates that a theoretically predicted yield of four moles of hydrogen is produced per one mole of glucose consumed).

In this application the term “hydrolysis” means the chemical reaction of a chemical, such as a carbohydrate, with water, resulting in the decomposition of the carbohydrate. In particular embodiments it may be catalyzed enzymatically or by other chemical catalysts, or by temperature or may be promoted by any suitable combinations of chemical or physical means.

In this application the term “nitrogen source” or “suitable nitrogen source” means any source of nitrogen that can be utilised by bacteria and includes organic and inorganic nitrogen compounds. Examples include: nitrates, nitrites, ammonium salts, amino compounds, proteins and amino acids and derivatives, compounds and complexes of the foregoing. Such nitrogen sources may be supplied in pure, semi pure, or mixed form and may be included as components of hydrolysed or unhydrolysed extracts of plants, animals, yeasts or other organisms.

In this application the term “pH” has its usual meaning as a measure of acidity or alkalinity and the term “pH controlled” means, with respect to an environment, that the pH of that environment is actively maintained or adjusted within a desired range. Such pH control may be maintained by chemical intervention (such as the addition of acid, alkali or buffers) or electrical intervention, or by any other suitable means.

In this application the terms “pure” and “substantially pure” have their normal meanings and encompass a mixed but defined population which is substantially pure of foreign elements. By way of example, substantially pure culture or mixture of bacterial species may include one or more designated bacterial species, so long at these are substantially pure of other, non-designated bacterial species. Hence a substantially pure culture of bacterial species of the genus Clostridium could include individuals from a number of Clostridium species, so long as it is substantially free of bacteria from other genera.

In this application, the term “sugar” has its normal meaning and includes any simple carbohydrate formed of monosaccharide units may be typified by a crystalline appearance and sweet taste and includes without limitation: straight, branched chain and ring forms; reducing and non-reducing forms; mono, di, tri, oligo and polysaccharides; trioses, pentoses and hexoses; alternative forms such as isomers, α and β forms, stereoisomers, and alternative chiral forms; ketoses, aldoses, dialdoses, diketoses, ketoaldoses, aldoketoses, aldosuloses, oxy and deoxy sugars, amino sugars, thio sugars and other biologically metabolisable substituted sugars; substituted, unsubstituted, saturated and unsaturated sugars. Examples may include but are not limited to glucose, galactose, xylose, fructose, sucrose, lactose, trehalose, cellobiose, gentiobiose, isomaltose, melibiose, primiverose, rutinose, maltose, pyranose, mannose, ribose, deoxyribose and compounds derived therefrom or comprising subunits thereof. It will be understood that the examples presented are not limiting and that any chemical satisfying the commonly understood or technical meanings of the word “sugar” may be useable in certain embodiments.

In this application “waste” means any source of carbon or sugars which may be generally unwanted, unusable or undesirable. It includes but is not limited to material which is not a primary desired product of an activity and is not produced primarily for sale or use. Such waste may, but need not necessarily, have a high carbohydrate content. Examples include but are not limited to municipal waste, industrial waste, domestic waste, agro-industrial waste, agricultural waste, farm waste, food processing waste, meat waste, paper waste, brewing waste, dairy waste; all types of plant material such as corn cobs, nut shells, husks, vegetable waste, fruit peels and seeds, plant waste, berry processing fiber, apple processing waste, raspberry processing waste; all types of dairy waste including spoilt milk, cheese and whey; brewing waste including yeast residues; pulp and paper mill waste, sugar cane bagasse, newspaper print fiber; cellulose, hemi-cellulose, fibrous waste, and may be hydrolysed or unhydrolysed. The term includes both treated and untreated waste and may include mixtures containing one or more of the foregoing. It will be understood that the foregoing types of waste are presented by way of example only and are not limiting, it will also be understood that there may be significant overlap between some of the different categories of waste set out.

In this application, the term “yield” means, with respect to hydrogen production, the number of moles of hydrogen liberated per mole of the relevant carbon source.

In a first embodiment there are disclosed bacteria of the genus Clostridium which can ferment a culture medium containing sugar and maintained under substantially anaerobic conditions to produce hydrogen.

In particular embodiments, the bacteria may be any strain from the genus Clostridium and may be present as one component of a mixed population of bacteria. In particular embodiments the bacteria may include Clostridium bifermentans or Clostridium difficile, and in certain embodiments may include IDA deposits (also and interchangeably referred to as “strains”, “isolates” etc. herein) KAM1, KAM5, KAM7, KAM8, KAM10, KAM 12 and KAM13 which are further identified, and whose IDA Accession Numbers are presented, in Table 8 hereof. In particular embodiments the bacteria may be single isolates or may comprise mixtures of two or more of the foregoing isolates, or may be descended from any of the foregoing isolates. They may be selected from existing ATCC deposits or from natural sources and their properties may be selected using any conventional procedures including the methods set out herein.

In particular embodiments hydrogen may be produced at an efficiency of anywhere between 34% and 95% and the efficiency may be greater than 34%, greater than 36%, greater than 38%, greater than 40%, greater than 42%, greater than 44%, greater than 46%, greater than 48%, greater than 50%, greater than 60%, greater than 62%, greater than 64%, greater than 66%, greater than 68%, greater than 70%, greater than 72%, greater than 74%, greater than 76%, greater than 78%, greater than 80% or greater than 90%.

Any sugar may be used under suitable conditions, and in certain embodiment glucose may be the substrate. In various alternative embodiments the substrate sugar may also be glucose, fructose, mamnose, galactose, sucrose, maltose, xylose, lactose, trehalose, cellobiose; gentiobiose; isomaltose; melibiose; primiverose; rutinose; pyranose; ribose; deoxyribose or other sugars or may be a mixture of two or more of the foregoing. The fermentation mixture may contain other components.

In particular embodiments the sugar may be present in the culture medium at a concentration of anywhere between 0.01% and 10%. In particular embodiments the sugar concentration may be in ranges of 0.01%-0.5%, 0.5%-1%, 1%-2%,2%-3%,3%,3%-4%,4%-5%,5-6%,6%-7%, 7%-8%,8%-9%, 9%-10%. It is hoped that with future improvements it may become possible to operate the process at sugar concentrations in excess of 10% such as 10%-15%, 15%-20% and 20%-30%.

The culture medium may generally have an initial pH of anywhere between about 4.5 and 8.5. In particular embodiments the initial pH may be between about 4.5 and 5.0, 5.0 and 5.5, 5.5 and 6.0, 6.0 and 6.5, 6.5 and 7.0, 7.0 and 7.5, 7.5 and 8.0, 8.0 and 8.5. In particular embodiments fermentation may be conducted with or without pH control of the fermentation mixture. In embodiments where the pH of the fermentation is controlled then it may be adjusted to and/or kept anywhere in the range of about 4.5 to 8.5. In particular embodiments the pH may be maintained to remain between about 4.5 and 5.0, 5.0 and 5.5, 5.5 and 6.0, 6.0 and 6.5, 6.5 and 7.0, 7.0 and 7.5, 7.5 and 8.0, 8.0 and 8.5 during part or all of the fermentation process.

The temperature of the fermentation is generally in the range between about 20° C. and 80° C. In particular embodiments it may be between about: 25° C. and 30° C., 30° C. and 35° C., 35° C. and 40° C., 40° C. and 45° C., 45° C. and 50° C., 50° C. and 55° C., 55° C. and 60° C., 60° C. and 65° C., 65° C. and 70° C., 70° C. and 75° C., or 75° C. and 80° C.

The reaction may be completely or substantially anaerobic. In particular embodiments the percentage of free oxygen in the fermentation mixture may be anywhere between 0% and 10% and may be less than 10%, less than 9%, less than 8%, less than 7%, less than 6%, less than 5%, less than 4%, less than 3%, less than 2%, less than 1%, less than 0.5%, less than 0.1%, or less than 0.01% of the available gases.

The fermentation mixture may contain a suitable nitrogen source or may contain no added nitrogen. In particular embodiments a range of suitable nitrogen sources may be used to supplement the fermentation mixture and in particular embodiments the final concentration of nitrogen may be adjusted to anywhere between 0.01% and 10%, such as 0.01%-0.5%, 0.5%-1%, 1%-2%,2%-3%,3%,3%-4%,4%-5%,5-6%,6-7%, 7%-8%, 8%-9%, 9%-10% of the culture medium.

In a second embodiment there are provided methods for the treatment of biomass comprising treating the biomass to liberate sugars and fermenting the sugars with a substantially pure culture of species of bacteria of the genus Clostridium maintained under substantially anaerobic conditions. In various embodiments the bacteria and the parameters of their use may be the same as those set out above. In particular embodiments the sugar and/or the fermentation mixture are derived from biomass of the types and using the methods set out herein.

In a third embodiment, the Clostridium bacteria described herein are used to ferment a culture medium comprising at least one sugar and maintained under substantially anaerobic conditions to produce hydrogen at an efficiency of at least about 34% of the maximum theoretically possible efficiency. In specific embodiments, the reaction conditions and results may be modified in the ways described above. In some embodiments, the sugar fermented may be a part of, may be mixed with, or may be derived from, biomass. Such sugar may be free in the raw biomass or may be liberated therefrom by pre-treatment of the biomass including pretreatment to hydrolyse the carbohydrates therein. This pretreatment, including any hydrolysis, may be by any conventional means. In certain embodiments the biomass may come from a range of sources as further set out herein and may comprise a variety of waste as also defined herein. In some embodiments it may be desirable to mechanically fragment the biomass to break up fibers and lumps to facilitate chemical or other pretreatment or to directly release sugars. In certain embodiments the biomass may have a high carbohydrate content by which is meant that the biomass contains a large proportional volume or weight of carbohydrate material.

In a fourth embodiment there is disclosed a substantially pure culture of one or more species of bacteria of the genus Clostridium able to ferment sugars present in a culture medium at a concentration between about 1% and about 10% under substantially anaerobic conditions to produce hydrogen with an efficiency of at least about 34% of maximum possible theoretically yield. In particular embodiments, the sugar may be glucose, lactose or other sugars. In various embodiments, the bacteria used and parameters of their use may be the same as those set out in other embodiments and in particular embodiments the sugar and/or the fermentation mixture may be derived from biomass of the types and using the methods set out herein.

Methods

To assess the behaviour of particular bacterial strains and to assess the effectiveness of fermentation, the fermentation mixture, containing biomass with or without nutrient supplementation, was sterilized at high temperature and pressure. Thereafter, the medium was allowed to cool to the desired temperature and an inoculum of the desired anaerobic bacteria was added. The fermentation temperature preferred was that which would facilitate the highest hydrogen productivity and was most suitable to the particular bacterial isolate or population. Experimentally, mesophilic conditions between 30° C. and 37° C. are often suitable but the temperature may be adjusted to suit the particular temperature requirements of other bacterial strains such as thermophilic strains.

Bacteria were isolated using normal methods and may be isolated in pure culture, co-cultures of two or more strains or as a mixture of species. They may be derived from defined culture collections, such as the ATCC, or may be collected from nature. The bacteria isolated were Clostridium species, and were either stock cultures deposited in culture collections or new isolates taken from natural environments. To obtain isolates of the appropriate genus, bacterial spores were selected by successive rounds of heat treatment and culturing to eliminate most strains other than the relatively heat tolerant Clostridia.

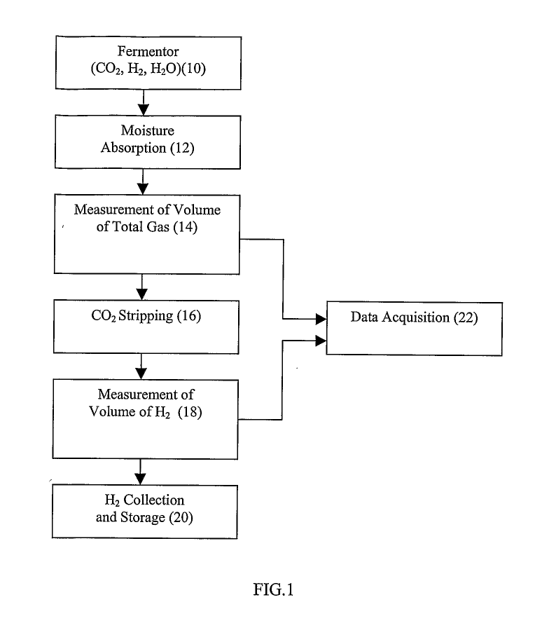

It will be appreciated that using the methods and bacteria disclosed herein, and with suitable adjustments, hydrogen may be produced at any production capacity from lab-scale to pilot plant, by varying fermentor and bioreactor configurations. For experimental purposes, hydrogen may be separated from carbon dioxide by first passing the fermentor effluent gas through an inline carbon dioxide scrubber. An easily accessible carbon dioxide scrubber can be provided by bubbling the fermentation gases through a 30% potassium hydroxide (w/v) solution. After separation, hydrogen may be collected in a multi-layered hydrogen-impermeable gas bag. With reference now to FIG. 1, in a typical experimental protocol gas is passed from the fermentor (10), through a suitable moisture absorber (12) where the gas is dried using suitable materials such as silica gel and anhydrous calcium sulfate, alternatively moisture may be removed by any conventional methods. Next the total volume of the gas is measured by a suitable device or method (14). This can be accomplished by standard techniques such as the use of a flow meter and following volumetric measurement, carbon dioxide is removed (16). This can be accomplished by using a range of conventional techniques such as passing the gas through potassium hydroxide solution. Following removal of carbon dioxide, the volume of hydrogen is measured (18) using conventional methods. After measurement the hydrogen is generally collected and stored (20) for use in any further analysis. Hydrogen production was compared to the total volume of evolved gases by suitable instruments or methods (22).

### EXAMPLES

### Example 1

### Hydrogen Production from Pulp Mill Waste Fiber

The feasibility of producing hydrogen from highly lignocellulosic pulp mill waste fiber was assessed to compare hydrogen production from a preferred substrate such as glucose with the production obtained from waste streams. Typically wood fiber may be made up of lignin (20-28%), cellulose (42-45%) and hemicellulose (27-30%). The hemicellulose and cellulose fractions may constitute about 70% of total wood mass but the proportion of each constituent varies depending on the source and type of wood. In many cases, cellulose may be embedded in a lignin matrix to constitute lignocellulose which is generally accepted to be a linear carbohydrate polymer consisting of repeating D (+) glucose units linked by β1→4 glucosidic bonds with the degree of polymerization depending on the source of the wood. These bonds can be hydrolyzed in strong acid conditions to release free glucose. Hemicellulose is also a carbohydrate, generally accepted to be made up of repeating units of xylose and may be amenable to dilute acid hydrolysis. Taken together these two fractions may provide a rich source of readily fermentable sugars upon hydrolysis. For the purpose of this example, pulp wood waste fiber was obtained from Norske Canada, Crofton, British Columbia.

For the production of hydrogen from pulp mill fiber the biomass was first mechanically fragmented to provide finer particle sizes, and sequentially or simultaneously treated by chemical or biological hydrolysis to release soluble sugars.

To 50 g of dried pulp mill waste fiber, 125 g of 72% sulfuric acid was added, and the resulting slurry was incubated at 30° C. for 2 h with constant stirring. The acid was adjusted to 20% (w/v) with deionized water and the solution autoclaved at 121° C. for 1 hour. The residue was separated by centrifugation at 4000 rpm for 10 minutes and re-extracted. The supernatants were pooled and neutralized by the addition of calcium hydroxide. The hydrolyzate fraction (supernatant) was then recovered from the solid fraction by centrifugation at 4000 rpm for 10 minutes The total sugar yield, as determined by the dinitrosalicylic acid (DNS) assay (Dubois, M., Gills, K. A., Hamilton, J. K., Rebers, P. A., and Smith, F., (1956) Colorimetric method for determination of sugars and related substances Anal. Chem. 28, 350-356) was 49% by weight of the original sample.

To assess the fermentability of the waste fiber hydrolyzate to hydrogen, the hydrolyzate fraction was diluted to 2% sugar content and supplemented with the following medium components per liter of deionized water: MgSO₄.7H₂O, 0.2 g; FeSO₄.7H₂O, 0.01 g; ρ-aminobenzoic acid, 0.1 g; yeast extract, 5 g; KH₂PO₄, 1.0 g; K₂HPO₄, 1.0 g; (NH₄)₂SO₄, 2.0 g; CH₃COONa, 6.56 g. The pH of the medium was adjusted to 7.5 with 5N KOH. For comparative purposes a control medium composed of 2% glucose as carbon source was supplemented with the above medium components. Fermentation was conducted in 250 ml media bottles at a working volume of 150 ml. Both media were sterilized by autoclaving at 120° C. for 20 minutes.

Clostridium beijerinckii ATCC 10132 was selected for biological hydrogen production. The microorganism was maintained in Peptone-yeast extract (PY) medium at room temperature. PY medium contained the following components per liter of deionized water: tryptone, 5.0 g; peptone, 5.0 g; yeast extract, 10.0 g; glucose, 5.0 g; beef extract, 5.0 g; Tween 80, 1.0 ml; K₂HPO₄, 2.0 g; salt solution, 40.0 ml; hemin solution, 10.0 ml; vitamin K₁, 200 μl; and L-cysteine.HCl.H₂O, 0.5 g. The salt solution comprised the following components per liter of deionized water: CaCl₂.2H₂O, 0.25 g; MgSO₄.7H₂O, 0.50 g; K₂HPO₄, 1.0 g; KH₂PO₄, 1.0 g; NaHCO3, 10.0 g; NaCl, 2.0 g. The pH of the medium was adjusted to 7.5 with 5N KOH. To ready this working culture for the production of hydrogen, 1 ml of the stock culture was transferred into 10 ml of fresh PY medium, heat-shocked at 75° C. for 15 minutes, and incubated at 37° C. overnight. Anaerobic conditions were maintained with the aid of a hydrogen and carbon dioxide generator in a BBL Gaspak™ jar (BBL, UK). The actively growing culture was used to inoculate the respective media to constitute 2% (vol/vol) of the total culture volume. Following inoculation, the culture headspace was sparged with nitrogen gas (99.9%) and all bottles were tightly closed with open-top screw-caps each fitted with a butyl septum and placed in the respirometer (Challenge Environmental Systems, Fayetteville, Ark.). Fermentation was carried out at a temperature of 37° C. and the cultures were agitated at 100 rpm. The culture pH was not controlled during fermentation.

Gas produced in the fermentor was channeled through a needle inserted through the butyl septum at the top of the bottle and directed to the inlet of a bubble counter for the measurement of total gas production. For the measurement of hydrogen produced, the effluent gas from the first bubble counter was passed through a 100 ml solution of 30% KOH contained in a 150 ml bottle inserted inline which stripped CO₂ from the gas stream. The residual gas was then channeled into a second bubble counter for the measurement of hydrogen gas. To ensure that the 30% KOH solution did indeed remove CO₂ from the gas stream, the H₂ content of the effluent gas was also measured with a hydrogen sensor (H₂Scan™ DCH, CA). Each bubble counter was pre-calibrated and linked through an interface to a computer. The volume of gas measured at any given time by a bubble counter was computed as real-time volumetric data and continuously logged in the computer. The parameters of culture performance were based on the rate of hydrogen production, as measured by volume of hydrogen produced, the yield (mol Hydrogen per mol carbon source) and the efficiency of conversion. Efficiency was defined as the actual hydrogen yield (in mol hydrogen per mol carbon source) expressed as a percentage of the theoretical yield of 4 mol hydrogen per mol glucose.

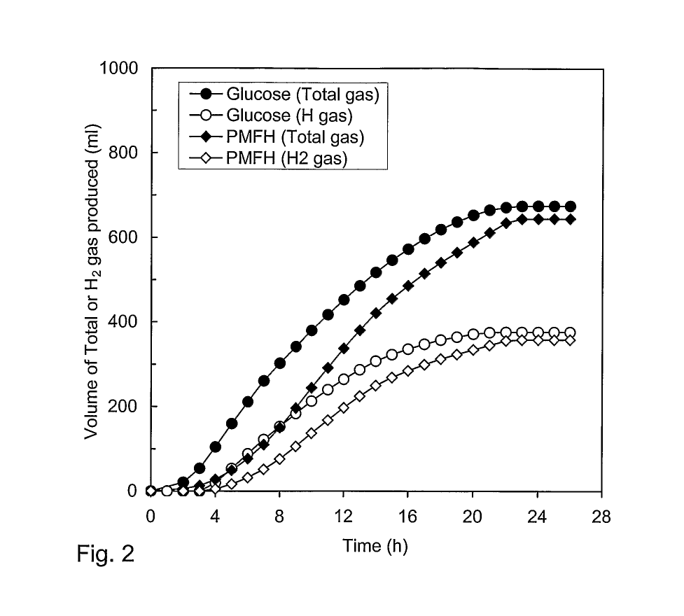

The production profiles of both total gas (carbon dioxide and hydrogen) and hydrogen alone were assessed during the course of fermentation in the glucose and pulp mill waste fiber hydrolyzate media. Referring to the results shown in FIG. 2, total gas production and hydrogen production were compared for C. beijerinckii (ATCC 10132) fermented on two carbon sources: pulp mill fiber hydrolyzate, PMFH (diamonds) and glucose (circles). Levels of total gas (solid symbols) and hydrogen gas (open symbols) were observed for both carbon sources. The cumulative volume of gas produced (ml) was measured over a 26 hour period. As will be seen, at the commencement of fermentation, the rate of hydrogen production was slightly higher from glucose than from the hydrolyzate of the pulp mill waste fiber.

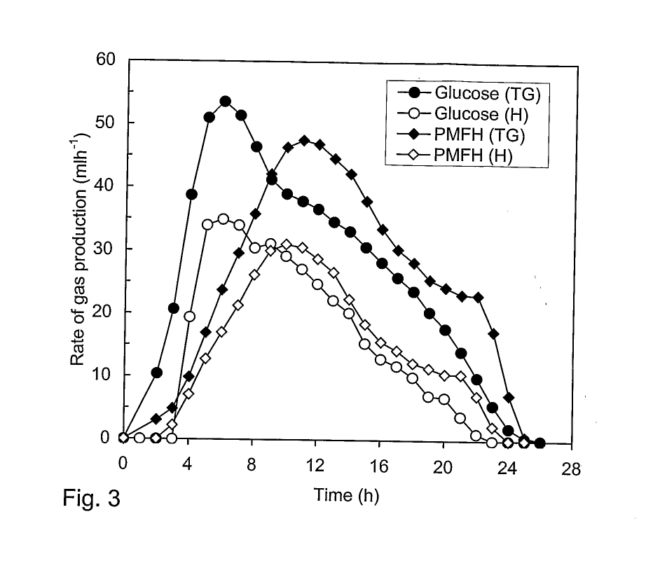

Referring now to FIG. 3, this shows the rate of total gas production measured as ml/hr. The closed symbols represent total gas produced and the open symbols represent hydrogen gas only. As will be seen, production rate peaked earlier for the glucose substrate (circles) than for the PMFH (diamonds) during a 26 hour fermentation period. This pattern was mirrored by the production of hydrogen gas where peak rate of hydrogen production for the hydrolyzed wood-pulp fermentations lagged behind that of glucose fermentations. The rates were comparable for both substrates. Although hydrogen production ceased after 23 hours but the fermentation was monitored for 26 hours.

Table 1 shows hydrogen production by Clostridium beijerinckii (ATCC 10132) on pulp mill fiber hydrolyzate (PMFH). As will be seen, hydrogen yield from the wood hydrolyzate was about 88% of that obtained from glucose.

TABLE 1

| | | | | Volume of | H₂ production | | | |
|---|---|---|---|---|---|---|---|---|
| Carbon | Total sugars (%) | | Duration | total gas | Volume | Yield | Productivity | Efficiency |
| source | Initial | Residual | (h) | (ml) | (ml) | (mol/mol) | (mmol/h) | (%) |
| PMFH | 2.0 | 0.020 | 23 | 588.8 | 334.4 | 0.90 | 0.649 | 22.6 |
| Glucose | 2.0 | 0.056 | 23 | 653.3 | 375.9 | 1.03 | 0.730 | 25.7 |

### Example 2

### Hydrogen Production from Cheese Whey

The feasibility of employing biomass streams from food and dairy processing for hydrogen production was assessed. They may contain readily fermentable sugars and may require little or no pretreatment for use in hydrogen production. Cheese whey makes up a large part of the volume of the total milk utilized in the cheese making process and contains a large part of the total solids in the original milk but has low levels of protein. Depending on the goals of the manufacturer, it may be discarded without further treatment (unskimmed whey), defatted (skimmed), or the residual proteins may be recovered and formulated into a protein-rich whey drink (deproteinized whey). Cheese whey is normally acidic having a pH in the range of 4.5 to 5.5. The levels of residual lactose, proteins and other components present in whey may vary according to the type of cheese manufactured. For the purpose of producing hydrogen, cheese whey may first be diluted such that the total sugar content is reduced to 2-3% (wt/vol) to reduce lactose repression of microbes during subsequent microbial fermentations. The fermentation mixture may be supplemented with an appropriate nitrogen source and the pH may be adjusted to suit the organism used. Generally an initial pH of 6.5 to 8.5 was found to be suitable. The sources of nitrogen for this fermentation can be selected from any of several known industrial sources including inorganic and organic nitrogen sources and yeast autolyzates produced from brewer's yeast sludge.

Two types of cheese whey (unskimmed and deproteinized whey) were obtained from local manufacturers and stored at 4° C. until use. The total sugar concentration was determined and the whey was diluted with water to give a final sugar content of 2% (w/v). The pH was adjusted to 7.5 with 5N KOH. The working volume varied between 100 and is 300 ml in either 250 or 500 ml media bottles depending on the microorganism used.

To investigate the effect of nutrient supplementation on hydrogen production, a parallel experiment was conducted with deproteinized whey as the primary carbon source. This was supplemented with the following components: tryptone, 6 g; yeast extract, 2 g; K₂HPO₄, 0.5 g; MgSO₄.7H₂O, 0.3 g; and FeSO₄.7H₂O, 50 mg per liter of deionize Control experiments were conducted using TY medium with lactose as the primary carbon source as it is the principal sugar in cheese whey. Clostridium saccharoperbutylacetonicum ATCC 27021, the microorganism used in the cheese whey fermentation studies, was maintained as spores in PY broth at room temperature. The microorganism was activated for fermentation by transferring 1 ml of this stock culture into 10 ml of fresh PY medium and heat-shocking at 75° C. for 15 min, followed by anaerobic incubation at 30° C. overnight with the aid of a hydrogen and carbon dioxide generator in a BBL Gaspak™ jar (BBL, UK). The growing culture was used to inoculate the respective media to constitute 2% of the total culture volume. Following inoculation, the culture headspace was sparged with nitrogen gas (99.9%) and all were bottles tightly closed with open-top screw-caps each fitted with a butyl septum and placed in the respirometer (Challenge Environmental Systems, Fayetteville, Ark.). Fermentation was carried out at a temperature of 30° C. and the cultures were agitated at 100 rpm. The culture pH was not controlled during fermentation. Hydrogen fermentation then proceeded in the respirometer as previously described. Fermentation was continued for up to 91 hours and the hydrogen production for three types of cheese whey were compared against glucose and lactose as the primary carbon source. Table 2 shows the results in the form of hydrogen production by Clostridium saccharoperbutylacetonicum ATCC 27021 from cheese whey (CW) which was either unskimmed (U), deproteinized (D), or deproteinized and supplemented with 200 ml TY medium components (DS).

TABLE 2

| | | | | Volume of | H₂ production | | | |
|---|---|---|---|---|---|---|---|---|
| Carbon | Total sugars (%) | | Duration | total gas | Volume | Yield | Productivity | Efficiency |
| source | Initial | Residual | (h) | (ml) | (ml) | (mol/mol) | (mmol/h) | (%) |
| Glucose | 2.0 | 0.060 | 17 | 931.4 | 494.4 | 1.36 | 1.298 | 34.1 |
| Lactose | 1.81 | 0.057 | 30 | 1082.7 | 537.4 | 3.12 | 0.810 | 38.1 |
| CW-U | 2.90 | 0.213 | 80 | 1609.4 | 573.8 | 2.17 | 0.320 | 27.1 |
| CW-D | 2.50 | 0.116 | 91 | 1279.4 | 492.6 | 2.10 | 0.242 | 26.3 |
| CW-DS | 2.62 | 0.093 | 28 | 1705.0 | 642.2 | 2.59 | 1.024 | 32.4 |

As will be seen, Clostridium saccharoperbutylacetonicum produced comparable yields of hydrogen from both types of unsupplemented cheese whey. The yield from unsupplemented cheese whey was between 67 and 83% of that obtained from lactose, and the rates of production were also correspondingly lower. The rate of hydrogen production was higher in the unskimmed whey than in the deproteinized whey. This may not be surprising as the deproteinized whey was very low in nitrogen content. Supplementation of the deproteinized whey with a suitable nitrogen source enhanced the rate of hydrogen production which resulted in a shorter fermentation time and a 23% increase in the hydrogen yield.

### Example 3

### Bioprospecting for Suitable Bacterial Isolates Capable of Hydrogen Production

New hydrogen-producing strains collected from natural environments were investigated. Clostridium species exposed to unfavorable environmental conditions form endospores which facilitate their survival. These spores are resistant to mild temperature treatment and only recommence vegetative growth at moderately high temperatures (Hyung et al., (1983) Ultrastructure and extreme heat resistance of spores from thermophilic Clostridium species. J. Bacteriol. 156, 1332-1337.). This property was exploited to enrich for spore-forming microorganisms from nature. Anaerobic cultivation techniques were used throughout this process. Soil samples were obtained from different environments in British Columbia including a bog, the submerged shoreline of a river, a seasonally flooded potato field and a flower garden.

For the germination of spores, 1-2 g of each soil sample was placed in 10 ml of a modified Peptone yeast extract (PY) medium in 25 ml screw-cap bottles, heat-shocked at 75-90° C. for 10 minutes and anaerobically incubated at 33° C. overnight in a BBL Gaspak jar (BBL, UK) with a hydrogen and carbon dioxide generator. This starter culture was then used to inoculate 200 ml of PY medium containing 2% glucose. The modified PY medium contained the following components per liter of deionized water: tryptone, 5.0 g; peptone, 5.0 g; yeast extract, 10.0 g; glucose, 5.0 g; beef extract, 5.0 g; Tween-80, 1.0 ml; K₂HPO₄, 2.0 g; salt solution, 40.0 ml; hemin solution, 10.0 ml; vitamin K₁, 200 μl; and L-cysteine.HCl.H₂O, 0.5 g; Nystatin, 5 mg; Cycloheximide, 5 mg. The salt solution comprised the following components per liter of deionized water: CaCl₂.2H₂O, 0.25 g; MgSO₄.7H₂O, 0.50 g; K₂HPO₄, 1.0 g; KH₂PO₄, 1.0 g; NaHCO3, 10.0 g; NaCl, 2.0 g. The pH of the medium was adjusted to 7.5 with 5N KOH and sterilized at 120° C. for 15 minutes Nystatin and cycloheximide were added to the medium prior to inoculation. These antifungal agents were incorporated into the media to retard fungal growth that may have been present in the soil samples.

Selection pressure to enrich for anaerobic bacterial species was applied through successive rounds of subculturing. There was continued selection for anaerobic strains by transferring 10% of each preceding culture into 200 ml of freshly prepared modified PY (as PYA) or TYA medium in a 250 ml bottle each containing 3 g/l of ammonium acetate and 2% glucose. Three rounds of subculturing was done in the PYA medium and a fourth conducted in TYA medium. The TYA medium contained the following components per litre of deionized water: tryptone, 6 g; yeast extract, 2 g; CH₃COONH₄, 3.0 g; K₂HPO₄, 0.5 g; MgSO₄.7H₂O, 0.3 g; and FeSO₄.7H₂O, 50 mg. All subculturing was conducted in the respirometer at 33° C. with agitation at 100 rpm as previously described. The mixed cultures were left to sporulate prior to each subsequent round of subculturing. The incidence and enrichment of hydrogen producers in the soil samples was detected by the evolution of hydrogen gas by the mixed cultures during the course of fermentation assays in the respirometer.

Four rounds of subculturing from the fermentation vessel and reinoculation into fresh media resulted in four cultures representing a mixed assemblage of microbes isolated from PFS (Potato field soil, Saanich Peninsula, Vancouver Island, B.C.), BS (Bog soil, Rithet's Bog, Saanich, Vancouver Island, B.C.), SSL (Submerged shoreline of Elk Lake, Vancouver Island, B.C.) and GS (garden soil, Surrey B.C.). Hydrogen production from these mixed assemblages was compared to that of the ATCC reference strain Clostridium saccharoperbutylacetonicum ATCC 27021. Referring now to Table 3, this shows hydrogen production by enriched microbial cultures in TYA medium.

TABLE 3

| Source of | | | | Volume of | H₂ production | | |
|---|---|---|---|---|---|---|---|
| mixed | Total glucose (%) | | Duration | total gas | Volume | Yield | Efficiency |
| culture | Initial | Residual | (h) | (ml) | (ml) | (mol/mol) | (%) |
| ATCC | 2.0 | 0.060 | 17 | 931.4 | 659.2 | 1.32 | 33.0 |
| PFS | 2.0 | 0.057 | 18 | 1082.7 | 376.7 | 0.94 | 23.6 |
| BS | 2.0 | 0.213 | 18 | 1609.4 | 553.5 | 1.39 | 34.6 |
| SSL | 2.0 | 0.116 | 18 | 1279.4 | 705.0 | 1.77 | 44.2 |
| GS | 2.0 | 0.093 | 18 | 1705.0 | 626.7 | 1.57 | 39.3 |

As will be seen, consistent selection pressure on the cultures followed by four rounds of subculturing in PYA and TYA media resulted in the enrichment of hydrogen-producing anaerobic bacterial species suitable for individual culturing. Heat enrichment and selection on sulfite iron and blood agar was used to isolate individual anaerobic hydrogen producers. As the mixed culture from the submerged shoreline of Elk Lake (SSL) yielded the highest efficiency (44.2%) of hydrogen production, single cultures were isolated by heat-shocking the mixed cultures as before followed by differentiation for sulfite-reducing clostridial spores in Sulfite Iron agar (SIA). Single black colonies (presumed to be clostridia) appearing in the SIA plates were excised and subcultured overnight in Reinforced Clostridial Medium (RCM). Cultures exhibiting good growth were inoculated into a modified PY medium containing 1.5 g/l ammonium acetate (as PYA medium) and the hydrogen potential determined using the respirometer assay as previously described. Clostridial species were then positively selected by streaking hydrogen-producing cultures on to Columbia CNA agar containing 5% sheep blood. Seven solitary colonies showing a zone of hemolysis on blood agar were picked, activated in RCM and their hydrogen potential retested in PYA medium using the respirometer assay as previously described.

All seven isolates used here were gram positive, anaerobic, motile, rod-like bacteria. They were oxidase and catalase negative, hydrolyzed starch and produced gas and acid from glucose and starch.

Hydrogen production of the seven natural isolates was compared to that of Clostridium saccharoperbutylacetonicum ATCC 27021 cultivated in 200 ml glucose fermentation medium. All the isolates produced hydrogen with a reasonable efficiency with glucose as a carbon source. Referring now to Table 4, this shows hydrogen production from glucose (PY medium) by SSL river soil isolates.

TABLE 4

| | | | | | | H₂ production | | |
|---|---|---|---|---|---|---|---|---|
| | Glucose (g/l) | | Final | Dur | Vol | Yield | Productivity | Efficiency |
| Isolate | Initial | Residual | pH | (h) | (ml) | (mol/mol) | (mmole/h) | (%) |
| KAM1 | 14.4 | 0.15 | 5.04 | 10 | 343.4 | 0.97 | 1.53 | 24.2 |
| KAM 5 | 14.4 | 0.00 | 5.13 | 17 | 542.4 | 1.51 | 1.42 | 37.8 |
| KAM 7 | 14.4 | 0.00 | 5.14 | 15 | 531.4 | 1.48 | 1.58 | 37.1 |
| KAM 8 | 14.4 | 0.00 | 5.09 | 15 | 546.7 | 1.53 | 1.63 | 38.1 |
| KAM 10 | 14.4 | 0.18 | 5.07 | 11 | 526.5 | 1.49 | 2.14 | 37.2 |
| KAM 12 | 14.4 | 3.68 | 5.13 | 14 | 497.4 | 1.86 | 1.59 | 46.6 |
| KAM 13 | 14.4 | 1.71 | 5.10 | 15 | 528.7 | 1.67 | 1.57 | 41.8 |
| ATCC | 10.0 | 0.00 | 5.49 | 12 | 319.0 | 1.28 | 1.13 | 32.0 |
| 27021 | 20.0 | 0.60 | 5.82 | 20 | 659.2 | 1.36 | 1.47 | 34.1 |

As will be seen, the rate of hydrogen production varied between 1.42 and 2.14 mmol/h when the initial glucose concentration was 14.4 g/l. The yield of a few isolates was considerably better than that of C. Saccharoperbutylacetonicum ATCC 27021 even when the latter was grown on an initial concentration of 20.0 g/l glucose. The hydrogen yield of all but isolate KAM1 were found to be in the range of 1.48 to 1.86 mole of hydrogen/mole of glucose, significantly higher than the 1.36 mole/mole of glucose generated by ATCC 27021. Isolate KAM12 demonstrated the highest efficiency of conversion of glucose to hydrogen gas.

To further assess the ability of these seven isolates to utilize agro-industrial waste products as a carbon source, hydrogen production was assessed in three types of cheese whey (unskimmed, skimmed and deproteinized). Unskimmed and skimmed whey were used without nutrient supplementation, whereas deproteinized whey was used with and without supplementation. When supplemented, the following tryptone-yeast extract acetate (TYA) medium components were added per liter of deionized water: tryptone, 6 g; yeast extract, 2 g; K₂HPO₄, 0.5 g; MgSO₄.7H₂O, 0.3 g; and FeSO₄.7H₂O, 50 mg. Fermentation was conducted in 250 ml bottles at a working volume of 200 ml. The initial pH of each medium was adjusted to 7.5 and sterilized. In each case, a 5% overnight inoculum of each culture was added to the cooled medium after sterilization. Following inoculation, the culture headspace was sparged with nitrogen gas (99.9%) and all were bottles tightly closed with open-top screw-caps each lifted with a butyl septum and placed in the respirometer (Challenge Environmental Systems, Fayetteville, Ark.). Fermentation was carried out at a temperature of 33° C. and the cultures were agitated at 50 rpm. The culture pH was not controlled during fermentation. Total gas and hydrogen gas production were measured as before.

All seven isolates produced hydrogen from all three types of cheese whey. Table 5 shows the results of fermentation in nutrient-supplemented deproteinized whey. The volume of hydrogen produced was variable. Although nutrient supplementation did enhance sugar consumption and volume of hydrogen production, none of the isolates completely consumed the sugar initially provided with approximately 40% of the sugar remaining. This may have been due to the drop of the final pH of the culture and a cessation of fermentation. A similar trend was observed when the isolates were cultivated in skimmed and unskimmed whey.

Referring to Table 5, this shows hydrogen production from nutrient-supplemented deproteinized cheese whey by SSL river soil isolates in 200 ml cultures.

TABLE 5

| | | | | | H₂ production | | | |
|---|---|---|---|---|---|---|---|---|
| | Total sugars (g/l) | | Final | Dur | Vol. | Yield | Productivity | Efficiency |
| Isolate | Initial | Residual | pH | (h) | (ml) | (mol/mol) | (mmole/h) | (%) |
| KAM 1 | 17.3 | 7.52 | 5.03 | 31 | 364.9 | 2.86 | 0.53 | 35.8 |
| KAM 5 | 17.3 | 6.47 | 4.90 | 31 | 491.4 | 3.48 | 0.71 | 43.5 |
| KAM 7 | 17.3 | 7.42 | 4.95 | 31 | 348.6 | 2.71 | 0.50 | 33.8 |
| KAM 8 | 17.3 | 7.04 | 4.88 | 31 | 421.0 | 3.15 | 0.61 | 39.3 |
| KAM 10 | 17.3 | 6.06 | 5.13 | 31 | 356.4 | 2.43 | 0.51 | 30.4 |
| KAM 12 | 17.3 | 6.43 | 4.84 | 31 | 275.7 | 1.94 | 0.40 | 24.3 |
| KAM 13 | 17.3 | 6.28 | 4.90 | 31 | 268.2 | 1.87 | 0.39 | 23.3 |

To counteract the effects of pH depression on efficiency of conversion of sugar content to hydrogen gas the culture pH was periodically adjusted to remain between pH 6.0 and 7.0 with supplemental alkali as shown in Table 6. Nutrient-supplemented deproteinized whey was inoculated with isolate KAM5. Medium supplementation and experimental conditions were as previously described for nutrient-supplemented deproteinized whey medium. The culture pH was adjusted at 21 hours and 53 hours post-inoculation using 5N KOH solution and hydrogen production monitored. Periodic pH adjustment enhanced both hydrogen production and sugar consumption. About 90% of the total sugar was consumed as compared 63% when pH was not adjusted. The volume of hydrogen produced was about 180% of that produced without pH adjustment, although the yield was similar. Moreover, the respective volume and yield of hydrogen produced by isolate KAM5 were about 120% and 165% of that produced by ATCC 27021.

Referring now to Table 6, this shows the effect of pH adjustment on hydrogen production from nutrient supplemented deproteinized cheese whey by river soil isolate KAM5 in 200 ml cultures. KAM5 indicates a culture wherein pH was not controlled. KAM5(pH) indicates that the pH of this culture was adjusted at 21 hours and 53 hours to lie between pH 6 and 7 during fermentation. These results indicated that KAM5 could produce hydrogen more efficiently than ATC 27021 when culture pH was regulated.

TABLE 6

| | | | | H₂ production | | | |
|---|---|---|---|---|---|---|---|
| | Total sugars (g/l) | | Duration | Volume | Yield | Productivity | Efficiency |
| Isolate | Initial | Residual | (h) | (ml) | (mol/mol) | (mmole/h) | (%) |
| KAM5 | 22.8 | 17.0 | 71 | 265.9 | 3.50 | 0.56 | 43.7 |
| KAM5 (pH) | 22.8 | 3.10 | 71 | 883.0 | 3.42 | 0.55 | 42.8 |
| ATCC | 26.2 | 0.93 | 28 | 732.1 | 2.20 | 1.17 | 27.6 |
| 27021 | | | | | | | |

### Example 4

### Hydrogen Production from Nutrient-Supplemented Apple Peel Waste by SSL River Soil Isolates

The feasibility of using apple peel was assessed. Apple peel used in this study was obtained from a local food processing company. To one kilogram of the waste, 1.25 L of deionized water was added and ground for 10 min in a laboratory warring blender. The mixture was filtered through cheesecloth into a clean container. The above procedure was repeated using the residue and 1.25 L of water. The filtrates were pooled together. The total volume of this fraction was 2.915 L and the total sugar concentration was 22.3 g/l. It was stored at 4° C. and used as the source of carbon in the fermentation study.

To assess the fermentability of the sugar solution to hydrogen by the seven isolates, it was diluted to yield a total sugar concentration of 2% (20 g/l) and supplemented with the following tryptone-yeast extract acetate (TYA) medium components per liter: tryptone, 6 g; yeast extract, 2 g; K₂HPO₄, 0.5 g; MgSO₄.7H₂O, 0.3 g; and FeSO₄.7H₂O, 50 mg. Fermentation was conducted in 250 ml bottles at a working volume of 200 ml. The initial pH of each medium was adjusted to 7.5 and it was sterilized. In each case, a 5% overnight inoculum of each culture was added to the cooled medium after sterilization. Following inoculation, the culture headspace was sparged with nitrogen gas (99.9%) and all bottles were tightly closed with open-top screw-caps each fitted with a butyl septum and placed in the respirometer (Challenge Environmental Systems, Fayetteville, Ark.). Fermentation was carried out at a temperature of 33° C. and the cultures were agitated at 50 rpm. The culture pH was not controlled during fermentation. However, to counteract the effect of pH depression, the culture pH was adjusted to lie between pH 6 and 7 after 44 h of cultivation with 5N KOH solution. Hydrogen gas production was measured as before.

The results of fermentation are given in Table 7. All seven isolates produced hydrogen from the apple peel medium. Five of the seven isolates consumed between 80% and 91% of the total sugar initially provided and the volume of hydrogen produced was variable. In most cases, however, the isolates produced hydrogen with the same efficiency as from glucose or better. An efficiency of 50.9% was obtained by isolate KAM8.

TABLE 7
Hydrogen production from nutrient-supplemented
apple peel waste by SSL river soil isolates

| | | | | | H₂ production | | | |
|---|---|---|---|---|---|---|---|---|
| | Total sugars (%) | | Final | Duration | Vol | Yield | Productivity | Efficiency |
| Isolate | Initial | Residual | pH | (h) | (ml) | (mol/mol) | (mmole/h) | (%) |
| KAM1 | 22.7 | 3.9 | 5.14 | 95 | 602.9 | 1.29 | 0.28 | 32.2 |
| KAM 5 | 22.7 | 2.1 | 5.00 | 85 | 758.2 | 1.48 | 0.40 | 37.0 |
| KAM 7 | 22.7 | 11.0 | 6.07 | 104 | 217.7 | 0.75 | 0.09 | 18.7 |
| KAM 8 | 22.7 | 3.7 | 5.06 | 77 | 962.3 | 2.03 | 0.56 | 50.9 |
| KAM 10 | 22.7 | 12.1 | 5.10 | 86 | 349.1 | 1.32 | 0.18 | 33.1 |
| KAM 12 | 22.7 | 4.0 | 5.10 | 86 | 909.7 | 1.95 | 0.47 | 48.9 |
| KAM 13 | 22.7 | 4.6 | 5.04 | 77 | 893.1 | 1.98 | 0.52 | 49.6 |

Culture volume 200 ml

### Example 5

### Bacterial Strains

Table 8 shows the tentatively established identities of a series of bacterial isolates used in the various embodiments disclosed, along with their IDA Accession Numbers, assigned by the Canadian International Depository Authority, National Microbiology Laboratory, Health Canada, 1015 Arlington Street, Winnipeg, Manitoba, Canada, R3E 3R2, at which the deposits were made. It will be appreciated that the precise identity of the different strains, although tentatively determined, remains to be fully confirmed.

TABLE 8
Bacterial strains

| Name of IDA | | |
|---|---|---|
| deposit | Tentative | IDA Accession Number and |
| (strain) | identification | date of deposit |
| KAM 1 | Clostridium bifermentans | 130204-05/Mar. 2, 2004 |
| KAM 5 | Clostridium difficile | 130204-06/Feb. 13, 2004 |
| KAM 7 | Clostridium sp. | 130204-07/Feb. 13, 2004 |
| KAM 8 | Clostridium sp. | 130204-08/Feb. 13, 2004 |
| KAM 10 | Clostridium bifermentans | 130204-09/Feb. 13, 2004 |
| KAM 12 | Clostridium bifermentans | 130204-10/Feb. 13, 2004 |
| KAM 13 | Clostridium sp. | 130204-11/Feb. 13, 2004 |

It will be understood that the embodiments and examples set out herein are presented by way of example only and do not limit the full scope of the claimed subject matter. In particular a range of modifications and variants will be readily apparent to those skilled in the art who will be able to make suitable adjustments to apply the claimed subject matter to different substrates, bacteria and reaction conditions. All such variants are understood to fall within the scope of the subject matter claimed.

1. A method for producing hydrogen comprising fermenting a culture medium comprising at least one sugar and maintained under substantially anaerobic conditions with a bacterium selected from the genus Clostridium to produce hydrogen with an efficiency of at least about 34% relative to the maximum theoretically possible yield.

2. The method according to claim 1 wherein said efficiency is at least about 38%.

3. The method according to claim 2 wherein said efficiency is at least about 42%.

4. The method according to claim 1 wherein said fermentation occurs at a temperature between about 30° C. and about 50° C.

5. The method according to claim 1 wherein said fermentation occurs at a temperature between about 50° C. and about 70° C.

6. The method according to a claim 1 wherein the mixture has an initial pH of between about 6.5 and about 8.5.

7. The method according to claim 1 wherein the sugar is present at a concentration of between about 0.5% and about 8.0%

8. The method according to claim 1 wherein the mixture is held at a pH of between about 6.5 and about 8.0 during the fermentation.

9. The method according to claim 1 wherein the culture medium is supplemented with a suitable nitrogen source.

10. (canceled)

11. The method according to claim 1 wherein said bacterium is selected from or descended from the bacterium of any of the group of bacteria consisting of IDA deposits KAM 1, KAM 5, KAM 7, KAM 8, KAM 10, KAM 12 and KAM 13.

12. The method according to claim 1 wherein said sugar is selected from the group consisting of maltose, sucrose, fructose, galactose, arabinose, lactose, glucose, xylose, mannose and mixtures thereof.

13. The method according to claim 1 wherein said mixture comprising sugars is derived from biomass.

14. The method according to claim 13 wherein the biomass has a high carbohydrate content

15. The method according to claim 13 wherein said biomass comprises agricultural waste, food processing waste, industrial waste, cellulose, lignocellulose, pulp and paper mill waste, newspaper print fiber, dairy waste, waste meat, corn cobs, nut shells, husks, sugar cane bagasse, agro-industrial waste fiber, spoilt milk, cheese, whey, apple processing waste, raspberry processing waste, brewing yeast residues, and mixtures of two or more thereof.

16-20. (canceled)

21. The method according claim 1 further comprising treating the biomass to liberate free sugars.

22. The method according to claim 21 comprising;
(a). enzymatically hydrolysing a component of the biomass; or
(b). acidically hydrolysing a component of the biomass; or
(c). enzymatically and acidically hydrolysing a component of the biomass to release free sugars.

23. The method according to claim 1 wherein said maintaining of said substantially anaerobic conditions comprises sparging the culture medium with nitrogen.

24. The method according to claim 1 wherein the sugar is glucose.

25. The method according to claim 1 wherein the sugar is lactose.

26. The method according to claim 1 wherein the bacterium is Clostridium difficile.

27. The method according to claim 1 wherein the bacterium is Clostridium bifermentans.

28. A method for the treatment of biomass comprising treating the biomass to liberate sugars and fermenting the sugars with a substantially pure culture of a bacteria of the genus Clostridium maintained under substantially anaerobic conditions to produce hydrogen with an efficiency of at least about 34% relative to the theoretical maximum possible yield.

29. The method according to claim 28 wherein said efficiency is at least about 38%.

30. The method according to claim 29 wherein said efficiency is at least about 42%.

31. The method according to claim 28 wherein said fermentation occurs at a temperature between 30° C. and 50° C.

32. The method according to claim 28 wherein said fermentation occurs at a temperature between 50° C. and 70° C.

33. The method according to claim 28 wherein the mixture has an initial pH of between about 6.5 and 8.5.

34. The method according to claim 28 wherein the mixture is held at a pH of between about 6.5 and 8.5 during the fermentation.

35. The method according to claim 28 wherein said sugar is present at a concentration of between about 0.2 and about 8.0%.

36. The method according to claim 28 wherein the culture medium is supplemented with a suitable nitrogen source.

37. (canceled)

38. The method according to claim 28 wherein said bacterium is selected from or descended from the bacterium of any of the group of bacteria consisting of IDA deposits KAM 1, KAM 5, KAM 7, KAM 8, KAM 10, KAM 12 and KAM 13.

39. The method according to claim 28 wherein said sugar is selected from the group consisting of maltose, sucrose, fructose, galactose, arabinose, lactose, glucose, xylose, mannose and mixtures thereof.

40. The method according to claim 28 wherein said mixture comprising sugars is derived from biomass.

41. The method according to claim 40 wherein the biomass has a high carbohydrate content.

42. The method according to claim 40 wherein said biomass comprises any one or more of agricultural waste food processing waste, industrial waste, cellulose, lignocellulose, pulp and paper mill waste, newspaper print fiber, dairy waste, waste meat, corn cobs, nut shells, husks, sugar cane bagasse, agro-industrial waste fiber spoilt milk, cheese, whey, apple processing waste, raspberry processing waste, brewing yeast residues, and mixtures of two or more thereof.

43-47. (canceled)

48. The method according to claim 28 further comprising treating the biomass to liberate free sugars.

49. The method according to claim 48 comprising;
(a) enzymatically hydrolysing a component of the biomass; or
(b) acidically hydrolysing a component of the biomass; or
(c) enzymatically and acidically hydrolysing a component of the biomass to release free sugars.

50. The method according to claim 47 further comprising mechanically fragmenting the biomass.

51. The method according to claim 28 wherein said maintaining of said substantially anaerobic conditions comprises sparging the culture medium with nitrogen.

52. The method according to claim 28 wherein the sugar is glucose.

53. The method according to claim 28 wherein the sugar is lactose.

54. The method according to claim 20 wherein the bacterium is Clostridium difficile.

55. The method according to claim 28 wherein the bacterium is Clostridium bifermentans.

56. A substantially pure culture of one or more species of bacteria of the genus Clostridium which can ferment glucose or lactose present in a culture medium at concentration of between about 1% and about 10% under substantially anaerobic conditions to produce hydrogen with an efficiency of at least about 34% of the maxiniun possible theoretical yield.

57. The culture according to claim 56 wherein said efficiency is at least about 38%.

58. The use culture according to claim 57 wherein said efficiency is at least about 42%.

59. The culture according to claim 56 wherein said fermentation occurs at a temperature between about 30° C. and about 50° C.

60. The culture according to claim 56 wherein said fermentation occurs at a temperature between about 50° C. and about 70° C.

61. The culture according claim 56 wherein the culture medium has an initial pH of between about 6.5 and about 8.5.

62. The culture according to claim 61 wherein the pH of the culture medium is held at a pH of between about 6.5 and about 8.0 during the fermentation reaction.

63. The culture according to claim 56 wherein the glucose is present in the mixture at a concentration of between about 0.5 and about 8.0%.

64. The culture according to claim 56 wherein the culture medium is supplemented with a suitable nitrogen source.

65. (canceled)

66. The culture according to claim 56 wherein said bacterium is selected from or descended from the bacterium of any of the group of bacteria consisting of IDA deposits KAM 1, KAM 5, KAM 7, KAM 8, KAM 10, KAM 12 and KAM 13.

67. The culture according to claim 56 wherein the culture is a substantially pure culture of Clostridium difficile.

68. The culture according to claim 56 wherein the culture is a substantially pure culture of Clostridium bifermentans.

69-106. (canceled)
